(19) United States
(12) Patent Application Publication
(10) Pub. No.: US 2012/0129884 A1
(43) Pub. Date: May 24, 2012

(54) PHOTOCHEMOTHERAPEUTIC HETEROCYCLIC AGENTS HAVING ANTIPROLIFERATIVE AND ANTINEOPLASTIC ACTIVITY

(75) Inventors: Francesco Dall'Acqua, Padova (IT); Girolamo Cirrincione, Palermo (IT); Paolo Barraja, Palermo (IT); Alessia Vador, Zelarino Venezia (IT)

(73) Assignee: UNIVERSITA DEGLI STUDI DI PADOVA, Padova (IT)

(21) Appl. No.: 13/387,810

(22) PCT Filed: Jul. 28, 2010
(86) PCT No.: PCT/IT2010/000337

§ 371 Date: Jan. 30, 2012

(30) Foreign Application Priority Data
Jul. 29, 2009 (IT) PD2009A000224

(51) Int. Cl. A61K 31/437; A61P 35/00; C07D 471/04

(52) U.S. Cl. 514/300; 546/84

(57) ABSTRACT

The present invention concerns the synthesis of new analogs of angelicins, pyrrolo [3,2-h]quinoline, for the treatment of pathologies having hyperproliferative character included those having neoplastic nature. The treatment is based on the combined action of pyrrolo [3,2-h]quinolines and UV-A light, through a clinical approach defined as PUVA (psoralen-UVA light). The most important feature of these compounds is that they exert their remarkable photoxicity without any DNA damage, which is the main origin of the side effects of the PUVA therapy.

### STATE OF ART

Psoralens 1 are naturally occurring or synthetic furocoumarins used in combination with UV-A light, commonly referred as PUVA therapy, in the treatment of human skin diseases with hyperproliferative and/or autoimmune character such as psoriasis, vitiligo and T-cell lymphoma. (F. Dall'Acqua, G. Viola, D. Vedaldi, Cellular and molecular target of psoralen. CRC Handbook of Organic Photochemistry and Photobiology (2004) W. M. Hoorspool, F. Lenci, ed CRC Press).

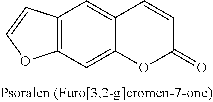

The photochemotherapy of psoriasis was introduced in clinical use in the early seventies at the Harvard Medical School of the Massachusetts General Hospital by oral administration of 8-methoxypsoralen (8-MOP) and subsequent exposure of the skin to UV-A irradiation. (J. A. Parrish, T. B. Fitzpatrick, M. A. Pathak, L. Tanenbaum, Photochemotherapy of psoriasis with oral methoxsalen and longwave ultraviolet light. N. Engl. J. Med. 1974, 291,1207-1211). Nowadays, PUVA therapy is commonly used in dermatology for the treatment of diseases such as vitiligo, psoriasis, plaque parapsoriasis, atopic dermatitis, generalized lichen planus, pigmentous urticaria and alopecia areata. (R. S. Stern, Psoralen and ultraviolet A light for therapy for psoriasis, N. Engl. J. Med. 2007, 357, 682-690; K. Danno, PUVA therapy: current concerns in Japan. J. Dermatol. Sci. 1999, 19, 85-105; R. Falabella, M. I. Barona, Update on skin repigmentation therapies in vitiligo, Pigment. Cell Melan. Res. 2008, 22, 42-65). In 1982, Eldelson et al. developed the extracorporeal photochemotherapy (ECP), photopheresis, using 8-MOP for the treatment of T-cell Cutaneous lymphoma. (R. Edelson, C. Berger, F. Gasparro, C. B. Jegasothy, P. Heald, B. Wintroub, E. Vonderheid, R. Knobler, K. Wolff, G. Plewig, G. McKiernan, I. Christiansen, M. Oster, H. Honigsmann, H. Wilford, E. Koroska, T. Rehle, G. Stingl, L. Laroche, Treatment of T-cell lymphoma by extracorporeal photochemotherapy. Preliminary results, N. Engl. J. Med. 1987, 316, 297-303). Thanks to photopheresis, which consists of the reinfusion of UV-A irradiated autologous leukocytes collected by apheresis and extracorporeally incubated with 8-MOP, the photochemotherapy of furocoumarins was used for the treatment of tumors (i. e. lymphoma). (L. Dalla Via, S. Marciani Magno, Photochemotherapy in the treatment of cancer, Curr. Med. Chem. 2001, 8, 1405-1418).

Furocoumarins are tricyclic aromatic compounds, which are capable to intercalate between nucleic acid base pairs thanks to their planar structure. Upon UV-A irradiation, they can covalently bind to DNA pirimidine bases. DNA adducts are formed by a [2+2]-photocycloaddition between one of the two photoreactive sites of psoralen (4′,5′ double bond of furan ring or 3,4 double bond of pyran ring) and the 5,6 double bond of thymine. The psoralen monoadducts formed can further photochemically react with a pyrimidine base of the complementary strand of the DNA, thus leading to interstrand cross links Kitamura, S. Kohtani, R. Nakagaki, Molecular aspects of furocoumarin reactions: Photophysics, Photochemistry, Photobiology and structural Analysis, J. Photochem. Photobiol. C: Photochem. Rev. 2005, 6, 168-185). The DNA interstrand cross links formation is the main cause for long term side effects of PUVA therapy, such as mutagenesis and the increased risk of skin cancer.

Preamble

PUVA therapy is efficacious in the treatment of dermatological and oncological diseases, however it exhibits some side effects, which limit its clinical use. These side effects may be divided in short term risks, which include nausea, skin phototoxicity and immunity depression and long term risks, such as premature skin aging, collagen degeneration of dermal and elastic tissues, cataract induction, but also mutagenicity and increased risk of neoplastic diseases. (J. A. Martin, S. Laube, C. Edwards, B. Gambles, A. V. Anstey, Rate of acute adverse events for narrow-band UVB and psoralen-UVA phototherapy, Photodermatol. Photoimmunol. Photomed. 2001, 23, 68-72; R. S. Stern, L. A. Thibodeau, R. A. Kleinerman, J. A. Parrish, T. B. Fitzpatrick, Risk of cutaneous carcinoma in patients treated with oral methoxsalen photochemotherapy for psoriasis, N. Engl. J. Med. 1979, 300, 809-813; K. T. Momtaz, T. B. Fitzpatrick, The benefits and risks of long-term PUVA photochemotherapy, Dermatol. Clint. 1998, 16, 227-34).

Attracted by the interesting field of photochemotherapy, many research group worked to obtain new derivatives that maintain the psoralen efficacy but devoid of side effects. In particular, the risk of skin neoplastic diseases could be avoided by using the angular furocoumarins, angelicin derivatives, which cannot give ICL for geometric reasons (Dall'Acqua, D. Vedaldi, A. Guiotto, P. Rodighiero, F. Carlassare, F. Baccichetti, F. Bordin, Methylangelicins: new potential agents for the photochemotherapy of psoriasis. Structure-activity studies on the dark and photochemical interactions with DNA, J. Med. Chem. 1981,24,806-811).

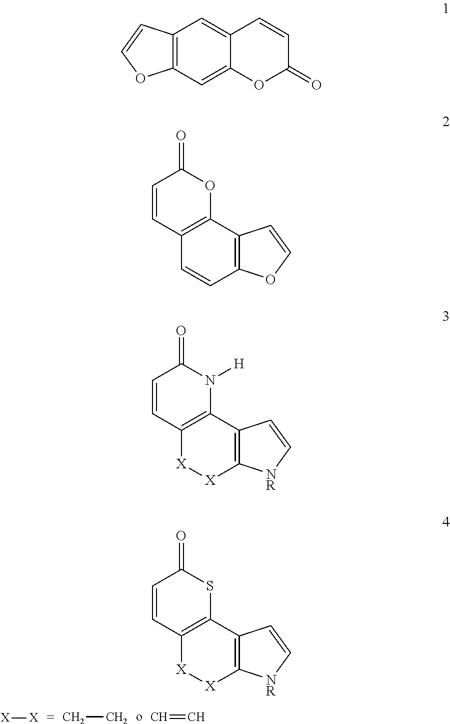

Another promising approach to dissociate undesired side effects from the therapeutic ones was the synthesis of heteroanalogues of psoralen 1 and angelicin 2. Thus, sulphur and nitrogen isosters such as thioangelicins, thienocoumarins, pyrrolocoumarins and furoquinolinones were studied and some of them showed improved interaction with DNA both in the dark and under UV-A irradiation in comparison to angelicin itself.

More recently, we reported the synthesis of the new ring systems pyrrolo[2,3-h]quinolinones 3 and thiopyrano[2,3-e]indolones 4, (P. Barraja, P. Diana, A. Lauria, A. Montalbano, A. M. Almerico, G. Dattolo, G. Cirrincione, G. Viola, F. Dall'Acqua, Pyrrolo[2,3-h]quinolinones: synthesis and photochemotherapic activity, Bioorg. Med. Chem. Lett. 2003, 13, 2809-2811; P. Barraja, P. Diana, A. Montalbano, G. Dattolo, G. Cirrincione, G. Viola, D. Vedaldi, F. Dall'Acqua, Pyrrolo[2,3-h]quinolinones: a new ring system with potent photoantiproliferative activity. Bioorg. Med. Chem. 2006, 14, 8712-8728; P. Barraja, L. Sciabica, P. Diana, A. Lauria, A. Montalbano, A. M. Almerico, G. Dattolo, G. Cirrincione, S. Disarò, G. Basso, G. Viola, F. Dall'Acqua, Synthesis and photochemotherapeutic activity of thiopyrano[2,3-e]indo1-2-ones. Bioorg. Med. Chem. Left. 2005, 15, 2291-2294; P. Barraja, P. Diana, A. Montalbano, A. Carbone, G. Cirrincione, G. Viola, A. Salvador, D. Vedaldi, F. Dall'Acqua, Thiopyrano[2,3-e]indol-2-ones: Angelicin heteroanalogues with potent photoantiproliferative activity. Bioorg. Med. Chem. 2008, 16, 9668-9683) respectively diaza and thio-aza analogues of angelicin 2.

| | R | R₁ | R₂ |
|---|---|---|---|
| 10a | SO₂Ph | H | SO₂Ph |
| 10b | H | H | SO₂Ph |
| 10c | SO₂Ph | H | CN |
| 10d | Me | H | SO₂Ph |
| 10e | Bn | H | SO₂Ph |
| 10f | Ph | H | SO₂Ph |
| 10g | BnpMe | H | SO₂Ph |
| 10h | BnpOMe | H | SO₂Ph |
| 10i | H | CO₂Et | SO₂Ph |
| 10j | Me | CO₂Et | SO₂Ph |
| 10k | Bn | CO₂Et | SO₂Ph |
| 10l | BnpMe | CO₂Et | SO₂Ph |
| 10m | BnpOMe | CO₂Et | SO₂Ph |

All derivatives of the latter ring systems showed photoantiproliferative activity in human tumor cell lines (MCF-7, Jurkat, K-562, LoVo) and keratinocytes (NCTC-2544), having IC₅₀ values at micromolar and sub-micromolar level (3, IC₅₀ 0.4-16.4 μM; 4, 0.2-14.8 μM) and a significant UV-A dose-dependent cytotoxicity. For several derivatives, such biological activity resulted higher than 8-MOP, 5-methoxypsoralen (5-MOP) and angelicin, used as reference drugs. However, studies of linear dichroism (LD) strongly suggested that the new derivatives did not efficaciously interact with DNA, excluding the macromolecule as a target for the new compounds, and indicating a different mechanism from that of the lead compound angelicin.

### AIMS OF THE INVENTION

On the basis of this preamble, we recall that the present invention concerns the synthesis of new photochemotherapeutic agents to use in the treatment of neoplastic diseases. Encouraged by our results and considering our interest towards chemistry of pyrroles and indoles, we planned to extend the study of photochemotherapeutic activity to the heterocyclic of the pyrrolo[3,2-h]quinoline ring system 9, to evaluate the effect on the photobiological activity of the different condensation of the pyrrole nucleus on the quinoline moiety.

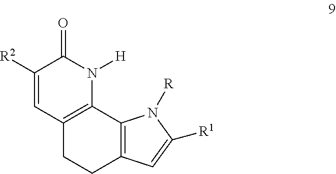

Moreover, the pyrrolo[3,2-h]quinoline ring system contains the quinolin-2-one moiety which confers to the molecule many biological properties. In fact, literature reported compounds incorporating the quinoline-2-one portion possessing potent antitumor activity (P. Rodighiero, A. Guiotto, A. Chilin, F. Bordin, F. Bacicchetti, F. Carlassare, D. Vedaldi, S. Caffieri, A. Pozzan, F. Dall'Acqua, Angular furoquinolinones, psoralen analogs: novel antiproliferative agents for skin diseases. Synthesis, biological activity, mechanism of action, and computer-aided studies. J. Med. Chem. 1996, 39, 1293-1302).

Another object of the invention comprehends the individuation of some compounds, among those synthesized, which present photoxic properties in vitro on some tumor cell lines comparable or superior to the activity of the reference compounds and which induce cell death via apoptosis with the involvement of both mitochondria and lysosomes.

Moreover, the invention comprehends the discovery of new photochemotherapeutic agents, which present a marked advantage in comparison with psoralens, as they do not interact with DNA neither photoinduce any type of damage to this macromolecule, excluding such a problem as the genotoxicity of the classic PUVA therapy.

Description of the Synthetic Strategy:

The synthetic pathway optimized for pyrrolo-quinolinones of type 3, starting from the corresponding tetrahydroindole-4-ones, has already pointed out the great versatility of the key enaminone intermediates which, could be utilized as reliable intermediates for the desired cyclization. (Barraja, P.; Diana, P.; Lauria, A.; Montalbano, A.; Almerico, A. M.; Dattolo, G.; Cirrincione, G.; Viola, G.; Dall'Acqua, F. Bioorg. Med. Chem. Lett. 2003, 13, 2809; Barraja, P.; Diana, P.; Montalbano, A.; Dattolo, G.; Cirrincione, G.; Viola, G.; Vedaldi, D.; Dall'Acqua, F. Bioorg. Med. Chem. 2006, 14, 8712).

Thus, tetrahydro-7H-indol-7-ones of type 6, in which it is possible to introduce the suitable functionality in a position to carbonyl group, would therefore represent the ideal building blocks for our synthesis. Such compounds can be prepared through known procedures. One of them, apparently convenient, led to tetrahydro-7H-indo1-7-one 6d, from 1,2-dioxocyclohexane and a commercially available acetal, but in low yield. (S. Massa, G. Stefancich, M. Artico, F. Corelli, R. Silvestri, Potential antitumor agents. Synthesis of pyrroloindazole derivatives related to the pyrroloindole moieties of the antitumor antibiotic CC-1065. Farmaco, Ed. Soc. 1987, 42, 567-574).

Instead, a more convenient multistep approach involved the annelation of the cyclohexanone moiety on the pyrrole ring. Thus, compounds 5a,b were obtained in excellent yields from the corresponding pyrrole derivative by an acylation with succinic anhydride followed by reduction. Cyclization in trifluoroacetic anhydride gave in 80-90% yield the tetrahydroindol-7-ones 6a,b (Scheme 1). (M. Kakushima, P. Hamel, R. Frenette, J. Rokach, Regioselective synthesis of acylpyrroles. J. Org. Chem. 1983, 48, 3214-3219. M. Tani, T. Ariyasu, M. Ohtsuka, T. Koga, Y. Ogawa, Y. Yokoyama, Y. Murakami, New strategy for indole synthesis from ethyl pyrrole-2-carboxylate (synthetic studies on indoles and related compounds. Chem. Pharm. Bull. 1996, 44, 55-61). The N-sulfonyl derivative 6a, upon heating in basic medium, allowed the isolation of the N-unsubstituted tetrahydroindol-7-one 6c, which, as well as the ethoxycarbonyl derivative 6b, can undergo nucleophilic reactions with alkyl or aralkyl halides to give N-substituted derivatives. Thus, reaction in tetrahydrofuran or dimethylformamide with methyl iodide or benzyl or substituted benzyl chlorides in the presence of sodium hydride, gave derivatives 6d,e,g-l in 70-90% yields. Instead, the N-phenyl derivative 6f was obtained in 66% yield by a modified Ulmann cross-coupling reaction. (J. J. Plattner, J. A. Parks, Preparation of new dihydrofuro[2,3-f]indole derivatives. J. Heterocyclic Chem. 1983, 20, 1059-1062).

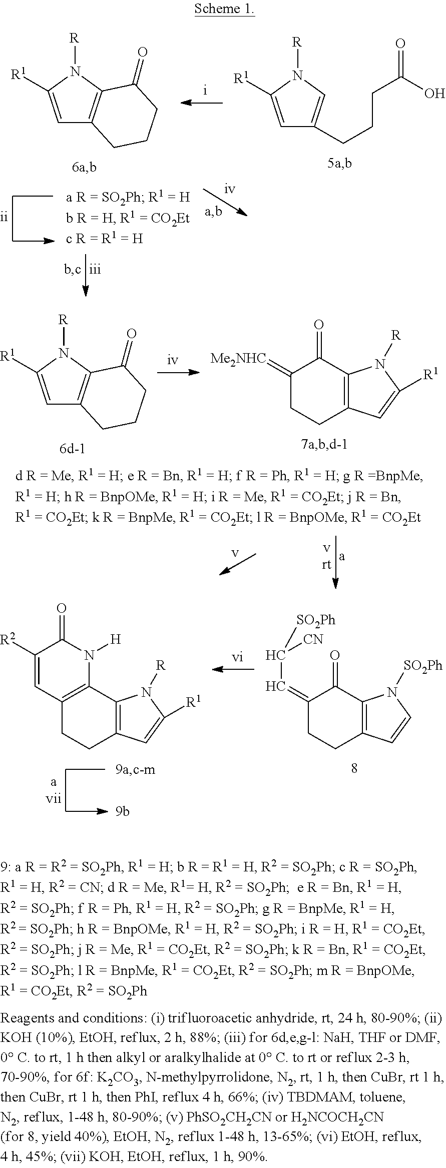

The variously substituted tetrahydroindoles 6a,b,d-l were reacted with the Bredereck reagent, t-butoxy-bis-(dimethylamino)methane (TBDMAM), to give the α-enaminoketons 7a,b,d-l in excellent yields (80-90%). (B. Stanovnik, J. Svete, Synthesis of Heterocycles from Alkyl 3-(Dimethylamino)propenoates and Related Enaminones. Chem. Rev. 2004, 104, 2433-2480). Compounds 7g,h,l were unstable and were utilized as crude products for the next step. Once we obtained the suitable substrates 7 to undergo the final cyclization, it was planned to react them with phenylsulfonylacetonitrile or cyanoacetamide as a C—C—N 1,3-dinucleophile to get the tricyclic derivatives 9. This choice resulted from the fact that in the pyrrolo-quinolinones of type 3, the presence of the phenylsulfonyl group in position 3 of the pyridone moiety was necessary to obtain compounds with good photoantiproliferative activity. Thus, reaction of the dimethylamino substituted derivatives 7a,b,d-l with phenylsulfonylacetonitrile in refluxing ethanol, under nitrogen atmosphere, gave the desired angelicin heteroanalogues 9a,d-m in acceptable yields (35-65%).

In the case of derivative 7a, when the reaction was performed at room temperature, a complex mixture was formed and it was possible to isolate the uncyclized intermediate 8, obtained in 40% yield. As the latter still possesses a cyano group, it strongly suggests that the reaction initiates with the nucleophilic attack of the methylene of the phenylsulfonylacetonitrile on the enaminone carbon, the most electrophilic site of compounds 7. Prolonged refluxing brings about the conversion of the nitrile moiety to carboxamide which, by nucleophilic attack to the ring carbonyl, cyclizes to the pyridone ring of the tricyclic system. Basic hydrolysis of the N-phenylsulfonyl derivative 9a led, in excellent yield, to the corresponding N-unsubstituted derivative 9b.The reaction of 7a with cyanoacetamide led to a very complex reaction mixture from which the 7-cyano substituted pyrroloquinolinone 9c was isolated in very poor yield (13%). Reaction of 7d with the same methylene active compound did not lead to the isolation of the corresponding tricyclic derivative. This behaviour was similar to that observed in the series of compounds 3 when the corresponding enaminones were reacted with cyanoacetamide. (Barraja, P.; Diana, P.; Lauria, A.; Montalbano, A.; Almerico, A. M.; Dattolo, G.; Cirrincione, G.; Viola, G.; Dall'Acqua, F. Bioorg. Med. Chem. Lett. 2003, 13, 2809; Barraja, P.; Diana, P.; Montalbano, A.; Dattolo, G.; Cirrincione, G.; Viola, G.; Vedaldi, D.; Dall'Acqua, F. Bioorg. Med. Chem. 2006, 14, 8712).

Examples reported below are provided as examples and do no limit anyway the significance of the invention.

### EXAMPLE 1

1-(Phenylsulfonyl)-1,4,5,6-tetrahydro-7H-indole-7-one (6a), ethyl 1,4,5,6,-tetrahydro-7H-indole-7-one-2-carboxylate (6b) and 1,4,5,6-tetrahydro-7H-indole-7-one (6c) were prepared as previously described and the white solids obtained showed spectroscopic data identical to those reported in literature (6a yield 85%; mp: 115-116° C.; 6b yield 82%; mp: 96-97° C.; 6c yield 85%; mp: 90-92° C.). (M. Kakushima, P. Hamel, R. Frenette, J. Rokach, Regioselective synthesis of acylpyrroles. J. Org. Chem. 1983, 48, 3214-3219. M. Tani, T. Ariyasu, M. Ohtsuka, T. Koga, Y. Ogawa, Y. Yokoyama, Y. Murakami, New strategy for indole synthesis from ethyl pyrrole-2-carboxylate (synthetic studies on indoles and related compounds. Chem. Pharm. Bull. 1996, 44, 55-61).

Preparation of 1-substituted 1,4,5,6-tetrahydro-7H-indole-7-ones (6d,e,g-l).

To a solution of the suitable ketone 6b or 6c (15 mmol) dissolved in anhydrous THF or DMF (20 mL), NaH (0.64 g, 16 mmole) was added at 0° C. and the reaction mixture was stirred at rt. After 1 h the suitable alkyl or aralkyl halide (16 mmol) was added at 0° C. and the reaction mixture was stirred at rt or refluxed for 2-4 h. Then, the reaction mixture was poured onto crushed ice and the precipitate was filtered off. In absence of precipitate the aqueous solution was extracted with dichloromethane (DCM, 3×50 mL) and the organic layers were separated, dried over sodium sulfate and the solvent removed in vacuo. Column chromatography of the residue, using DCM as eluent, gave the expected product.

1-Methyl-1,4,5,6-tetrahydro-7H-indole-7-one (6d).

This compound was obtained from the reaction of 6c with iodomethane in THF after 3 h at rt: brown oil; yield 90%; IR: ν 1635 (CO) cm ⁻¹; ¹H NMR: δ1.89-2.01 (2H, m, CH₂), 2.35 (2H, t, J=6.0 Hz, CH₂), 2.65 (2H, t, J=6.0 Hz, CH₂), 3.82 (3H, s, CH₃), 5.96 (1H, d, J=2.5 Hz, H-3), 7.03 (1H, d, J=2.5 Hz, H-2); ¹³C NMR: δ 23.3 (t), 24.8 (t), 35.9 (q), 38.7 (t), 106.3 (d), 126.2 (s), 130.7 (d), 136.8 (s), 187.7 (CO). Anal calcd for C₉H₁₁NO: C, 72.46; H, 7.43; N, 9.39. Found: C, 72.56; H, 7.69; N, 9.30.

1-Benzyl-1,4,5,6-tetrahydro-7H-indole-7-one (6e).

This compound was obtained from the reaction of 6c with benzylchloride in DMF after 2 h at rt: brown oil; yield 90%; IR: ν 1637 (CO) cm⁻¹; ¹H NMR: δ 1.88-2.01 (2H, m, CH₂), 2.35 (2H, t, J=6.0 Hz, CH₂), 2.68 (2H, t, J=6.0 Hz, CH₂), 5.49 (2H, s, CH₂), 6.05 (1H, d, J=2.5 Hz, H-3), 7.12-7.34 (6H, m, Ar and H-2); ¹³C NMR: δ 23.4 (t), 24.8 (t), 38.8 (t), 50.8 (t), 107.2 (d), 125.5 (s), 126.9 (d), 127.2 (d), 128.4 (d), 130.4 (d), 137.5 (s), 138.9 (s), 187.7 (CO). Anal calcd for C₁₅H₁₅NO: C, 79.97; H, 6.71; N, 6.22. Found: C, 80.30; H, 7.00; N, 6.03.

1-(p-Methyl-benzyl)-1,4,5,6-tetrahydro-7H-indole-7-one (6g).

This compound was obtained from the reaction of 6c with p-methyl-benzylchloride in DMF after 2 h at rt: white solid; yield 75%; mp: 80-82° C.; IR: ν 1637 (CO) cm⁻¹; ¹H NMR: δ 1.88-2.00 (2H, m, CH₂), 2.24 (3H, s, CH₃), 2.35 (2H, t, J=6.3 Hz, CH₂), 2.67 (2H, t, J=6.3 Hz, CH₂), 5.43 (2H, s, CH₂), 6.03 (1H, d, J=2.5 Hz, H-3), 7.05 (2H, d, J=7.5 Hz, H-3″ and H-5″), 7.10 (2H, d, J=7.5 Hz, H-2″ and H-6″), 7.20 (1H, d, J=2.5 Hz, H-2); ¹³C NMR: δ 20.6 (q), 23.4 (t), 24.8 (t), 38.8 (t), 50.6 (t), 107.1 (d), 125.4 (s), 127.1 (d), 128.9 (d), 130.2 (d), 135.8 (s), 136.4 (s), 137.4 (s), 187.7 (CO). Anal calcd for C₁₆H₁₇NO: C, 80.30; H, 7.16; N, 5.85. Found: C, 80.60; H, 7.06; N, 5.50.

1-(p-Methoxy-benzyl)-1,4,5,6-tetrahydro-7H-indole-7-one (6h).

This compound was obtained from the reaction of 6c with p-methoxy-benzylchloride in DMF after 2 h at rt: white solid; yield 75%; mp: 82-84° C.; IR: ν 1637 (CO) cm⁻¹; ¹H-INMR: δ 1.90-2.00 (2H, m, CH₂), 2.36 (2H, t, J=6.0 Hz, CH₂), 2.66 (2H, t, J=6.0 Hz, CH₂), 3.70 (3H, s, CH₃), 5.40 (2H, s, CH₂), 6.02 (1H, d, J=2.5 Hz, H-3), 6.85 (2H, d, J=7.5 Hz, H-3″ and H-5″), 7.16 (2H, d, J=7.5 Hz, H-2″ and H-6″), 7.20 (1H, d, J=2.5 Hz, H-2); ¹³C NMR: δ 23.4 (t), 24.8 (t), 38.8 (t), 50.2 (t), 54.9 (q), 107.1 (d), 113.7 (d), 125.4 (s), 128.6 (d), 130.1 (d), 130.8 (s), 137.4 (s), 158.5 (s), 187.7 (CO). Anal calcd for C₁₆H₁₇NO₂: C, 75.27; H, 6.71; N, 5.49. Found: C, 75.18; H, 7.02; N, 5.68.

Ethyl 1-methyl-1,4,5,6-tetrahydro-7H-indole-7-one-2-carboxylate (6i).

This compound was obtained from the reaction of 6b with iodomethane in DMF after 2 h under reflux: brown solid; yield 88%; mp: 51-52° C.; IR: ν 1707 (CO), 1651 (CO) cm⁻¹; ¹H NMR: δ 1.29 (3H, t, J=7.1 Hz, CH₃), 1.91-2.03 (2H, m, CH₂), 2.47 (2H, t, J=6.1 Hz, CH₂), 2.68 (2H, t, J=6.1 Hz, CH₂), 4.13 (3H, s, CH₃), 4.26 (2H, q, J=7.1 Hz, CH₂), 6.71 (1H, s, H-3); ¹³C NMR: δ 14.1 (q), 22.8 (t), 24.1 (t), 34.1 (q), 39.6 (t), 60.4 (t), 114.0 (d), 127.5 (s), 129.9 (s), 134.6 (s), 160.4 (CO), 190.1 (CO). Anal calcd for C₁₂H₁₅NO₃: C, 65.14; H, 6.83; N, 6.33. Found: C, 64.90; H, 7.10; N, 6.68.

Ethyl 1-benzyl-1,4,5,6-tetrahydro-7H-indole-7-one-2-carboxylate (6j).

This compound was obtained from the reaction of 6b with benzylchloride in DMF after 3 h at rt: brown solid; yield 70%; mp: 84-85° C.; IR: ν 1709 (CO), 1655 (CO) cm⁻¹; ¹H NMR: δ 1.21 (3H, t, J=7.1 Hz, CH₃), 1.93-2.05 (2H, m, CH₂), 2.48 (2H, t, J=6.0 Hz, CH₂), 2.74 (2H, t, J=6.0 Hz, CH₂), 4.20 (2H, q, J=7.1 Hz, CH₂), 6.01 (2H, s, CH₂), 6.84 (1H, s, H-3), 6.93-7.31 (5H, m, Ar); ¹³C NMR: δ 13.9 (q), 22.9 (t), 24.1 (t), 39.6 (t), 48.7 (t), 60.5 (t), 99.4 (s), 115.1 (d), 126.0 (d), 126.8 (d), 128.3 (d), 130.6 (s), 135.9 (s), 139.5 (s), 160.5 (CO), 190.0 (CO). Anal calcd for C₁₈H₁₉NO₃: C, 72.71; H, 6.44; N, 4.71. Found: C, 72.45; H, 6.69; N, 4.75.

Ethyl 1-(p-methyl-benzyl)-1,4,5,6-tetrahydro-7H-indole-7-one-2-carboxylate (6k).

This compound was obtained from the reaction of 6b with p-methyl-benzylchloride in DMF after 2 h at rt: white solid; yield 73%; mp: 65-66° C.; IR: ν 1710 (CO), 1651 (CO) cm⁻¹; ¹H NMR: δ 1.22 (3H, t, J=7.1 Hz, CH₃), 1.92-2.04 (2H, m, CH₂), 2.22 (3H, s, CH₃), 2.47 (2H, t, J=6.0 Hz, CH₂), 2.72 (2H, t, J=6.0 Hz, CH₂), 4.20 (2H, q, J=7.1 Hz, CH₂), 5.96 (2H, s, CH₂), 6.82 (1H, s, H-3), 6.85 (2H, d, J=8.0 Hz, H-3″ and H-5″), 7.06 (2H, d, J=8.0 Hz, H-2″ and H-6″); ¹³C NMR: δ 14.0 (q), 20.5 (q), 22.9 (t), 24.1 (t), 39.6 (t), 48.4 (t), 60.5 (t), 115.0 (d), 126.0 (d), 127.1 (s), 128.8 (d), 129.7 (s), 135.4 (s), 135.8 (s), 135.9 (s), 160.2 (CO), 190.0 (CO). Anal calcd for C₁₉H₂₁NO₃: C, 73.29; H, 6.80; N, 4.50. Found: C, 73.45; H, 6.70; N, 4.24.

Ethyl 1-(p-methoxy-benzyl)-1,4,5,6-tetrahydro-7H-indole-7-one-2-carboxylate (6l).

This compound was obtained from the reaction of 6b with p-methoxy-benzylchloride in DMF after 2 h at rt: yellow oil; yield 75%; IR: ν 1711 (CO), 1651 (CO) cm ⁻¹; ¹H NMR: δ 1.23 (3H, t, J=7.1 Hz, CH₃), 1.95-2.04 (2H, m, CH₂), 2.47 (2H, t, J=5.9 Hz, CH₂), 2.72 (2H, t, J=5.9 Hz, CH₂), 3.69 (1H, s, CH₃), 4.22 (2H, q, J=7.1 Hz, CH₂), 5.92 (2H, s, CH₂), 6.77-6.97 (5H, m, Ar and H-3); ¹³C NMR: δ 14.0 (q), 22.9 (t), 24.1 (t), 39.6 (t), 47.9 (t), 54.9 (q), 60.5 (t), 113.7 (d), 115.1 (d), 127.1 (s), 127.6 (d), 129.6 (s), 130.7 (s), 135.4 (s), 158.2 (s), 160.3 (CO), 190.0 (CO) Anal calcd for C₉H₂₁NO₄: C, 69.71; H, 6.47; N, 4.28. Found: C, 70.02; H, 6.70; N, 4.14.

Preparation of 1-phenyl-1,4,5,6-tetrahydro-7H-indole-7-one (6f).

To a solution of 6c (1.3 g, 9.63 mmol) in N-methyl-pyrrolidone (19 mL), K₂CO₃ (2.0 g, 14.45 mmol) was added under nitrogen atmosphere and the reaction mixture was stirred at rt for 1 h. Then CuBr (2.8 g, 19.26 mmol) was added and the reaction stirred for 1 h and finally iodobenzene (4.0 ml, 35.63 mmol) was added and the reaction refluxed for 4 h. After cooling, HCl (5%, 17 mL) was added to the reaction mixture and stirred for 1 h, then AcOEt (25 mL) was added and stirred for further 30 min. The reaction mixture was filtered through celite and washed with AcOEt (25 mL). The organic layer was shaken for 1 h with ice and NaCl, separated, dried over sodium sulfate and the solvent removed in vacuo to give a brown solid; yield 66%; mp: 66-68° C.; IR: ν 1650 (CO) cm⁻¹; ¹H NMR: δ 1.96-2.09 (2H, m, CH₂), 2.39 (2H, t, J=6.1 Hz, CH₂), 2.76 (2H, t, J=6.1 Hz, CH₂), 6.23 (1H, d, J=2.6 Hz, H-3), 7.23-7.45 (6H, m, Ar and H-2); ¹³C NMR: δ 23.5 (t), 24.5 (t), 38.9 (t), 108.6 (d), 125.5 (d), 126.1 (s), 127.0 (d), 128.4 (d), 131.1 (d), 130.1 (s), 139.7 (s), 186.3 (CO). Anal calcd for Cᵢ₄H₁₃NO: C, 79.59; H, 6.20; N, 6.63. Found: C, 79.30; H, 6.00; N, 6.80.

Preparation of 1-substituted 6-[(dimethylamino)methylene]-1,4,5,6-tetrahydro-7H-indole-7-one (7a,b,d-l).

To a solution of 6a,b,d-l (5.3 mmol) in anhydrous toluene (10 mL) TBDMAM (3.31 mL, 16 mmol)as added under nitrogen atmosphere and the reaction mixture was refluxed. After cooling, the precipitate was filtered and shaken in diethyl ether (25 mL), filtered and air dried.

6-[(Dimethylamino)methylene]-1-(phenylsulfonyl)-1,4,5,6-tetrahydro-7H-indole-7-one (7a). This product was obtained from 6a after 4 h reflux and precipitated as yellow solid; yield 90%; mp: 116-118° C.; IR: ν 1637 (CO) cm⁻¹; ¹H NMR (CDCl₃): δ 2.58 (2H, t, J=6.5 Hz, CH₂), 2.84 (2H, t, J=6.5 Hz, CH₂), 3.00 (6H, s, 2×CH₃), 6.16 (1H, d, J=2.7 Hz, H-3), 7.27 (1H, s, CH), 7.37-7.52 (3H, m, Ar), 7.61 (1H, d, J=2.7 Hz, H-2), 8.05 (2H, d, J=7.2 Hz, H-2′ and H-6′); ¹³C NMR (CDCl₃): δ 23.5 (t), 24.2 (t), 43.4 (q), 102.9 (s), 109.8 (d), 127.8 (d), 127.9 (d), 128.5 (d), 130.3 (s), 133.1 (d), 138.1 (s), 139.6 (s), 148.6 (d), 176.6 (CO). Anal calcd for C₁₇H₁₈N₂O₃S: C, 61.80; H, 5.49; N, 8.48. Found: C, 62.10; H, 5.26; N, 8.20.

Ethyl 6-[(dimethylamino)methylene]-1,4,5,6-tetrahydro-7H-indole-7-one-2-carboxylate (7b).

This product was obtained from 6b after 2 h reflux and precipitated as yellow solid; yield 80%; mp: 209-211° C.; IR: ν 3421 (NH), 1703 (CO), 1635 (CO) cm⁻¹; ¹H NMR: δ 1.27 (3H, t, J=7.1 Hz, CH₃), 2.58 (2H, t, J=6.7 Hz, CH₂), 2.88 (2H, t, J=6.7 Hz, CH₂), 3.07 (6H, s, 2×CH₃), 4.21 (2H, q, J=7.1 Hz, CH₂), 6.60 (1H, s, H-3), 7.36 (1H, s, CH), 12.01 (1H, s, NH); ¹³C NMR: δ 14.2 (q), 22.1 (t), 24.2 (t), 43.2 (q), 59.9 (t), 102.7 (s), 112.6 (d), 125.1 (s), 129.8 (s), 132.8 (s), 148.2 (d), 160.3 (CO), 177.7 (CO). Anal calcd for C₁₄H₁₈N₂O₃: C, 64.10; H, 6.92; N, 10.68. Found: C, 64.46; H, 7.26; N, 10.30.

6-[(Dimethylamino)methylene]-1-methyl-1,4,5,6-tetrahydro-7H-indole-7-one (7d).

This product was obtained from 6d after 4 h reflux and precipitated as brown solid; yield 90%; mp: 49-51° C.; IR: ν 1633 (CO) cm⁻¹; 6 2.55 (2H, t, J=6.4 Hz, CH₂), 2.83 (2H, t, J=6.4 Hz, CH₂), 3.02 (6H, s, 2×CH₃), 3.83 (1H, s, CH₃), 5.87 (1H, d, J=2.5 Hz, H-3), 6.86 (1H, d, J=2.5 Hz, H-2), 7.23 (1H, s, CH); ¹³C NMR: δ 23.0 (t), 24.7 (t), 35.8 (q), 43.1 (q), 103.5 (s), 105.3 (d), 127.3 (s), 128.6 (d), 131.8 (s), 146.6 (d), 178.4 (CO). Anal calcd for Cᵢ₂H₁₆N₂O: C, 70.56; H, 7.90; N, 13.71. Found: C, 70.30; H, 7.66; N, 13.42.

1-Benzyl-6[(dimethylamino)methylene]-1,4,5,6-tetrahydro-7H-indole-7-one (7e).

This product was obtained from 6e after 48 h reflux and precipitated as brown solid; yield 80%; mp: 116-118° C.; IR: ν 1633 (CO) cm⁻¹; ¹H NMR: δ 2.57 (2H, t, J=6.8 Hz, CH₂), 2.83 (2H, t, J=6.8 Hz, CH₂), 3.02 (6H, s, 2×CH₃), 5.56 (2H, s, CH₂), 5.94 (1H, d, J=2.5 Hz, H-3), 7.02 (1H, d, J=2.5 Hz, H-2), 7.12-7.28 (6H, m, Ar and CH); ¹³C NMR: δ 23.0 (t), 24.6 (t), 43.1 (q), 50.5 (t), 103.4 (s), 106.2 (d), 126.7 (s), 126.9 (d), 127.0 (d), 128.0 (d), 128.2 (d), 132.3 (s), 139.6 (s), 147.0 (d), 178.3 (CO). Anal calcd for C₁₈H₂₀N₂O: C, 77.11; H, 7.19; N, 9.99. Found: C, 77.00; H, 6.94; N, 10.15.

6-[(Dimethylamino)methylene]-1-phenyl-1,4,5,6-tetrahydro-7H-indole-7-one (7f).

This product was obtained from 6f after 24 h reflux and precipitated as brown solid; yield 80%; mp: 129-131° C.; IR: ν 1637 (CO) cm⁻¹; ¹H NMR: δ 2.63 (2H, t, J=6.4 Hz, CH₂), 2.91 (2H, t, J=6.4 Hz, CH₂), 3.02 (6H, s, 2×CH₃), 6.13 (1H, d, J=2.7 Hz, H-3), 7.08 (1H, d, J=2.7 Hz, H-2), 7.18 (1H, s, CH), 7.24-7.42 (5H, m, Ar); ¹³C NMR: δ 23.2 (t), 24.2 (t), 43.1 (q), 102.9 (s), 107.6 (d), 125.0 (d), 126.3 (d), 127.7 (s), 128.2 (d), 128.9 (d), 134.2 (s), 140.3 (s), 147.0 (d), 176.6 (CO). Anal calcd for C₁₇H₁₈N₂O: C, 76.66; H, 6.81; N, 10.52. Found: C, 77.00; H, 7.05; N, 10.17.

6-[(Dimethylamino)methylene]-1-(p-methylbenzyl)-1,4,5,6-tetrahydro-7H-indole-7-one (7g), 6-[(dimethylamino)methylene]-1-(p-methoxybenzyl)-1,4,5,6-tetrahydro-7H-indole-7-one (7h), ethyl 1-(p-methoxybenzyl)-6-[(dimethylamino)methylene]-1,4,5,6-tetrahydro-7H-indole-7-one-2-carboxylate (7l).

These compounds, obtained from 6g, 6h, 6I respectively after 3 h reflux as brown oils, were unstable and were utilized for the successive step without purification.

Ethyl 6-Rdimethylamino)methylenej-1-methyl-1,4,5,6-tetrahydro-7H-indole-7-one-2-carboxylate (7i).

This product was obtained from 6i after 3 h reflux and precipitated as brown solid; yield 80%; mp: 50-52° C.; IR: ν 1707 (CO), 1631 (CO) cm ⁻¹; ¹H NMR: δ 1.27 (3H, t, J=7.1 Hz, CH₃), 2.54 (2H, t, J=7.3 Hz, CH₂), 2.76 (2H, t, J=7.3 Hz, CH₂), 3.08 (6H, s, 2×CH₃), 4.17 (3H, s, CH₃), 4.23 (2H, q, J=7.1 Hz, CH₂), 6.66 (1H, s, H-3), 7.39 (1H, s, CH); ¹³C NMR: δ 14.2 (q), 22.4 (t), 24.1 (t), 33.9 (q), 43.3 (q), 59.9 (t), 103.7 (s), 113.6 (d), 125.4 (s), 129.6 (s), 132.2 (s), 148.8 (d), 160.5 (CO), 178.8 (CO). Anal calcd for C₁₅H₂₀N₂O₃: C, 65.20; H, 7.30; N, 10.14. Found: C, 65.54; H, 7.12; N, 10.05.

Ethyl 1-benzyl-6-[(dimethylamino)methylene]-1,4,5,6-tetrahydro-7H-indole-7-one-2-carboxylate (7j).

This product was obtained from 6j after 1 h reflux and precipitated as yellow solid; yield 85%; mp: 101-103° C.; IR: ν 1703 (CO), 1635 (CO) cm⁻¹; ¹H NMR: δ 1.20 (3H, t, J=7.0 Hz, CH₃), 2.61 (2H, t, J=6.1 Hz, CH₂), 2.83 (2H, t, J=6.1 Hz, CH₂), 3.06 (6H, s, 2×CH₃), 4.16 (2H, q, J=7.0 Hz, CH₂), 6.13 (2H, s, CH₂), 6.78 (1H, s, H-3), 6.92-7.25 (5H, m, Ar), 7.38 (1H, s, CH); ¹³C NMR: δ 14.1 (q), 22.3 (t), 24.0 (t), 43.3 (q), 48.2 (t), 59.9 (t), 103.5 (s), 114.7 (d), 125.0 (s), 126.0 (d), 126.5 (d), 128.2 (d), 130.2 (s), 131.9 (s), 139.7 (s), 149.1 (d), 160.3 (CO), 178.6 (CO). Anal calcd for C₂₁H₂₄N₂O₃: C, 71.57; H, 6.86; N, 7.95. Found: C, 71.84; H, 7.01; N, 7.63.

Ethyl 1-(p-methyl-benzyl)-6-[(dimethylamino)methylene]-1,4,5,6-tetrahydro-7H-indole-7-one-2-earboxylate (7k).

This product was obtained from 6k after 4 h reflux and precipitated as yellow solid and purified on a silica pad (DCM); yield 80%; mp: 84-86° C.; IR: ν 1709 (CO), 1631 (CO) cm⁻¹; ¹H NMR: δ 1.22 (3H, t, J=7.1 Hz, CH₃), 2.56-2.68 (4H, m, 2×CH₂), 2.64 (3H, s, CH₃), 3.04 (6H, s, 2×CH₃), 4.22 (2H, q, J=7.1 Hz, CH₂), 6.04 (2H, s, CH₂), 6.82 (2H, d, J=7.9 Hz, Ar), 6.87 (1H, s, H-3), 7.05 (2H, d, J=7.9 Hz, Ar), 7.61 (1H, s, CH); ¹³C NMR: δ 14.0 (q), 21.6 (q), 21.8 (t), 43.2 (q), 48.2 (t), 60.3 (t), 113.5 (s), 115.0 (d), 126.0 (d), 128.8 (d), 131.0 (s), 133.1 (s), 135.8 (s), 136.2 (s), 152.6 (d), 160.2 (CO), 180.01 (CO). Anal calcd for C₂₂H₂₆N₂O₃: C, 72.11; H, 7.15; N, 7.64. Found: C, 72.40; H, 7.38; N, 7.30.

Preparation of 1,7-disubstituted 1,4,5,9-tetrahydro-8H-pyrrolo[3,2-h]quinolin-8-one (9a-m).

To a suspension of 7a,b,d-l (4 mmol) in anhydrous ethanol (50 mL), the suitable cyanomethylene compound (6 mmol) in anhydrous ethanol (60 mL) was added dropwise under nitrogen atmosphere. After the addition the reaction mixture was refluxed. Upon cooling, a precipitate formed which was filtered and purified by recrystallization or by column chromatography (Sepacore Büchi) using DCM/AcOEt 9:1 as eluent.

1,7-bis(Phenylsulfonyl)-1,4,5,9-tetrahydro-8H-pyrrolo[3,2-h]quinolin-8-one (9a).

This product was obtained from the reaction of 7a with phenylsulfonylacetonitrile after 48 h reflux. The yellow solid was recrystallized from ethanol; yield 65%; mp: 349-351° C.; IR: ν 3218 (NH), 1635 (CO) cm⁻¹; ¹H NMR: δ 2.70-2.86 (4H, m, 2×CH₂), 6.83 (1H, d, J=2.0 Hz, H-3), 7.54-7.71 (7H, m, Ar and H-2), 7.91-7.98 (4H, m, Ar), 8.21 (1H, s, H-6), 11.90 (1H, s, NH); ¹³C NMR: δ 20.4 (t), 25.1 (t), 99.5 (d), 111.1 (s), 113.5 (d), 123.6 (s), 124.9 (s), 126.6 (d), 127.9 (d), 128.4 (s), 128.8 (d), 129.8 (d), 131.8 (s), 133.2 (d), 133.7 (d), 140.4 (s), 141.3 (s), 143.9 (d), 156.3 (CO). Anal calcd for C₂₃H₁₈N₂O₅S₂: C, 59.21; H, 3.89; N, 6.00. Found: C, 59.00; H, 4.01; N, 5.89. When the same reaction was carried out at rt was obtained a complex mixture which, after column chromatography (eluent DCM/EtOAc 9:1), furnished intermediate 8 as brown oil.

1-Phenylsulfonyl-6-[2-(phenylsulfonyl)-propanenitrile]-1,4,5,6-tetrahydro-7H-indole-7-one (8).

Yield 40%; IR: ν 2362 (CN), 1693 (CO) cm⁻¹ ; ¹H NMR: δ 2.53-2.72 (4H, m, 2×CH₂), 5.86 (1H, d, J=10.7 Hz, CH), 6.02 (1H, d, J=10.7 Hz, CH), 6.36 (1H, d, J=2.6 Hz, H-3), 7.15-8.08 (11H, m, Ar and H-2); ¹³C NMR: δ 23.7 (t), 34.3 (t), 111.0 (d), 125.5 (d), 128.1 (d), 128.4 (d), 128.5 (d), 128.8 (s), 129.2 (s), 129.3 (d), 131.6 (d), 133.3 (d), 134.4 (s), 134.6 (d), 136.8 (d), 137.6 (s), 147.7 (s), 143.7 (s), 162.8 (CO). Anal calcd for C₂₃H₁₈N₂O₅S₂: C, 59.21; H, 3.89; N, 6.00. Found: C, 59.04; H, 4.12; N, 6.12.

By refluxing in ethanol this intermediate, the cyclized compound 9a, in 45% yield, was obtained.

1-(Phenylsulfonyl)-1,4,5,9-tetrahydro-8H-pyrrolo[3,2-h]quinolin-7-carbonitrile-8-one (9c).

This product was obtained from the reaction of 7a with cyanoacetamide after 32 h reflux. The yellow solid precipitated was recrystallized from ethanol; yield 13%; mp: 309-311° C.; IR: ν 3309 (NH), 2220 (CN), 1653 (CO) cm⁻¹; NMR: δ 2.69-2.92 (4H, m, 2×CH₂), 6.85 (1H, s, H-3), 7.61-7.77 (4H, m, Ar and H-2), 7.96 (2H, d, J=8.0 Hz, H-2′ and H-6′), 8.04 (1H, s, H-6), 12.06 (1H, s, NH); ¹³C NMR: δ 20.3 (t), 25.0 (t), 99.5 (d), 112.2 (s), 113.5 (d), 117.0 (s), 125.0 (s), 126.7 (d), 128.3 (s), 129.8 (d), 131.9 (s), 133.8 (d), 138.8 (s), 141.3 (s), 148.4 (d), 159.5 (CO). Anal calcd for C₁₈H₁₃N₃O₃S: C, 61.53; H, 3.73; N, 11.96. Found: C, 61.32; H, 3.60; N, 12.20.

1-Methyl-7-(phenylsulfonyl)-1,4,5,9-tetrahydro-8H-pyrrolo[3,2-h]quinolin-8-one (9d).

This product was obtained from the reaction of 7d with phenylsulfonylacetonitrile after 24 h reflux. The yellow solid was recrystallized from ethanol; yield 55%; mp: 208-209° C.; IR: ν 3320 (NH), 1635 (CO) cm⁻¹; ¹H NMR: δ 2.51 (2H, t, J=6.9 Hz, CH₂), 2.82 (2H, t, J=6.9 Hz, CH₂), 3.94 (3H, s, CH₃), 6.00 (1H, d, J=2.4 Hz, H-3), 6.93 (1H, d, J=2.4 Hz, H-2), 7.54-7.72 (3H, m, Ar), 7.92 (2H, d, J=8.0 Hz, H-2′ and H-6′), 8.03 (1H, s, H-6), 11.47 (1H, s, NH); ¹³C NMR: S 21.6(t), 27.4 (t), 36.2 (q), 99.5 (d), 102.3 (s), 106.6 (d), 117.4 (s), 124.6 (s), 127.6 (d), 128.0 (d), 128.8 (d), 129.3 (s), 133.0 (d), 141.2, (s), 141.3 (s), 157.9 (CO). Anal calcd for C₁₈Hᵢ₆N₂O₃S: C, 63.51; H, 4.74; N, 8.23. Found: C, 63.72; H, 4.95; N, 8.02.

1-Benzyl-7-(phenylsulfonyl)-1,4,5,9-tetrahydro-8H-pyrrolo[3,2-h]quinolin-8-one (9e).

This product was obtained from the reaction of 7e with phenylsulfonylacetonitrile after 24 h reflux. The solid precipitated was purified by column chromatography (Sepacore Büchi) using DCM/AcOEt 9:1 as eluent. Brown solid; yield 45%; mp: 213-214° C.; IR: ν: 3214 (NH), 1631 (CO) cm⁻¹; ¹H NMR: δ 2.65 (2H, t, J=7.0 Hz, CH₂), 2.83 (2H, t, J=7.0 Hz, CH₂), 5.79 (2H, s, CH₂), 6.06 (1H, d, J=2.5 Hz, H-3), 7.09 (1H, d, J=2.5 Hz, H-2), 7.12-7.28 (5H, m, Ar), 7.54-7.71 (3H, m, Ar), 7.90-7.95 (2H, m, Ar), 8.00 (1H, s, H-6), 11.61 (1H, s, NH); ¹³C NMR: δ 21.6 (t), 27.5 (t), 50.8 (t), 99.5 (d), 107.3 (d), 114.7 (s), 120.9 (s), 124.4 (s), 126.9 (d), 127.1 (d), 127.7 (d), 128.3 (d), 128.9 (d), 129.7 (s), 133.1 (d), 137.2 (s), 139.3 (s), 141.2 (d), 150.9 (s), 158.1 (CO). Anal calcd for C₂₄H₂₀N₂O₃S: C, 69.21; H, 4.84; N, 6.73. Found: C, 69.46; H, 4.63; N, 6.50.

1-Phenyl-7-(phenylsulfonyl)-1,4,5,9-tetrahydro-8H-pyrrolo[3,2-h]quinolin-8-one (9f).

This product was obtained from the reaction of 7f with phenylsulfonylacetonitrile after 32 h reflux. The crude precipitate was purified by column chromatography (Sepacore Büchi) using DCM/AcOEt 9:1 as eluent. Yellow solid; yield 55%; mp: 251-252° C.; IR: ν 3359 (NH), 1654 (CO) cm⁻¹; ¹H NMR: δ 2.68 (2H, t, J=6.7 Hz, CH₂), 2.79 (2H, t, J=6.7 Hz, CH₂), 6.34 (1H, d, J=2.7 Hz, H-3), 7.29 (1H, d, J=2.7 Hz, H-2), 7.32-7.67 (8H, m, Ar), 7.90-7.96 (2H, d, J=8.0 Hz, H-2′ and H-6′), 8.15 (1H, s, H-6), 10.10 (1H, s, NH); ¹³C NMR: δ 22.0 (t), 26.5 (t), 99.5 (d), 108.6 (s), 109.5 (d), 112.1 (s), 119.0 (s), 121.6 (s), 124.5 (d), 127.4 (d), 127.9 (d), 128.7 (d), 129.3 (d), 130.9 (d), 133.0 (d), 133.6 (s), 138.6 (s), 140.9 (s), 156.4 (CO). Anal calcd for C₂₃H₁₈N₂O₃S: C, 68.64; H, 4.51; N, 6.96. Found: C, 68.40; H, 4.80; N, 7.06.

1-(p-Methyl-benzyl)-7-(phenylsulfonyl)-1,4,5,9-tetrahydro-8H-pyrrolo[3,2-h]quinolin-8-one (9g).

This product was obtained from the reaction of 7g with phenylsulfonylacetonitrile after 32 h reflux. The crude precipitate was purified by column chromatography (Sepacore Büchi) using DCM/AcOEt 9:1 as eluent. Yellow solid; yield 40%; mp: 178-180° C.; IR: ν 3217 (NH), 1626 (CO) cm⁻¹; ¹H NMR: δ 2.19 (3H, s, CH₃), 2.64 (2H, t, J=7.5 Hz, CH₂), 2.80 (2H, t, J=7.5 Hz, CH₂), 5.71 (2H, s, CH₂), 6.04 (1H, d, J=2.5 Hz, H-3), 7.02-7.10 (5H, m, Ar and H-2), 7.55-7.67 (3H, m, Ar), 7.90-7.95 (2H, m, Ar), 8.00 (1H, s, H-6), 11.56 (1H, s, NH); ¹³C NMR: δ 20.6 (q), 21.6 (t), 50.7 (t), 99.5 (d), 105.2 (d), 107.2 (d), 124.6 (s), 126.9 (d), 127.7 (d), 128.8 (d), 128.9 (d), 129.8 (s), 133.1 (d), 136.2 (s), 137.2 (s), 140.5 (s), 141.2 (s), 152.5 (s),158.0 (s), 174.0 (CO). Anal calcd for C₂₅H₂₂N₂O₃S: C, 69.75; H, 5.15; N, 6.51. Found: C, 69.55; H, 5.02; N, 6.64.

1-(p-Methoxy-benzyl)-7-(phenylsulfonyl)-1,4,5,9-tetrahydro-8H-pyrrolo[3,24]quinolin-8-one (9h).

This product was obtained from the reaction of 7h with phenylsulfonylacetonitrile after 32 h reflux. The crude precipitate was purified by column chromatography (Sepacore Büchi) using DCM/AcOEt 9:1 as eluent. Yellow solid; yield 45%; mp: 154-156° C.; IR: ν 3315 (NH), 1635 (CO) cm⁻¹; ¹H NMR: δ 2.64 (2H, t, J=7.5 Hz, CH₂), 2.78 (2H, t, J=7.5 Hz, CH₂), 3.34 (3H, s, CH₃), 5.68 (2H, s, CH₂), 6.04 (1H, d, J=2.5 Hz, H-3), 6.80 (2H, d, J=8.0 Hz, H-3″ and H-5″), 7.08 (1H, d, J=2.5 Hz, H-2), 7.12 (2H, d, J=8.0 Hz, H-2″ and H-6″), 7.56-7.68 (3H, m, Ar), 7.93 (2H, d, J=8.6 Hz, H-2′ and H-6′), 8.00 (1H, s, H-6), 11.59 (1H, s, NH); ¹³C NMR: δ 21.6 (t), 27.5 (t), 50.3 (t), 54.9 (q), 99.5 (d), 107.2 (d), 113.6 (d), 114.4 (s), 121.1 (s), 126.7 (d), 128.4 (d), 128.9 (d), 129.6 (s), 131.1 (s), 133.1 (d), 133.7 (d), 136.8 (s), 141.2 (s), 145.0 (s), 158.0 (s), 158.4 (CO). Anal calcd for C₂₅H₂₂N₂O₄S: C, 67.25; H, 4.97; N, 6.27. Found: C, 67.05; H, 5.18; N, 6.00.

Ethyl 7-(phenylsulfonyl)-1,4,5,9-tetrahydro-8H-pyrrolo[3,2-h]quinolin-8-one-2-carboxylate (9i).

This product was obtained from the reaction of 7b with phenylsulfonylacetonitrile after 32 h reflux. The yellow solid precipitated was recrystallized from ethanol; yield 60%; mp: 322-323° C.; IR: ν 3280 (NH), 3246 (NH), 1711 (CO), 1651 (CO) cm⁻¹; ¹H NMR: δ 1.30 (3H, t, J=7.1 Hz, CH₃), 2.67-2.88 (4H, m, 2×CH₂), 4.29 (2H, q, J=7.1 Hz, CH₂), 6.73 (1H, s, H-3), 7.55-7.72 (3H, m, Ar), 7.98 (2H, d, J=8.0 Hz, H-2′ and H-6′), 8.21 (1H, s, H-6), 12.06 (2H, bs, 2×NH); ¹³C NMR: δ 14.2 (q), 20.5 (t), 25.4 (t), 60.3 (t), 110.8 (s), 113.4 (d), 122.8 (s), 123.9 (s), 126.1 (s), 127.9 (d), 128.4 (s), 128.7 (d), 133.1 (d), 140.6 (2×s), 143.8 (d), 156.5 (CO), 159.8 (CO). Anal calcd for C₂₀H₁₈N₂O₅S: C, 60.29; H, 4.55; N, 7.03. Found: C, 59.98; H, 4.68; N, 6.85.

Ethyl 1-methyl-7-(phenylsulfonyl)-1,4,5,9-tetrahydro-8H-pyrrolo[3,2-h]quinolin-8-one-2-carboxylate (9j).

This product was obtained from the reaction of 7i with phenylsulfonylacetonitrile after 28 h reflux. The crude precipitate was purified by column chromatography (Sepacore Büchi) using DCM/AcOEt 9:1 as eluent. Yellow solid; yield 52%; mp: 214-216° C.; IR: ν 3120 (NH), 1703 (CO), 1643 (CO) cm⁻¹; ¹H NMR: δ 1.27 (3H, t, J=7.1 Hz, CH₃), 2.66 (2H, t, J=6.5 Hz, CH₂), 2.87 (2H, t, J=6.5 Hz, CH₂), 4.22 (2H, q, J=7.1 Hz, CH₂), 4.28 (1H, s, CH₃), 6.78 (1H, s, H-3), 7.57-7.74 (3H, m, Ar), 7.98 (2H, d, J=8.0 Hz, H-2′ and H-6′), 8.17 (1H, s, H-6), 11.96 (1H, s, NH); ¹³C NMR: δ 14.2 (q), 20.9 (t), 27.2 (t), 34.8 (q), 59.8 (t), 99.9 (d), 114.7 (d), 117.4 (s), 125.7 (s), 126.8 (s), 127.8 (d), 129.0 (d), 130.8 (s), 133.3 (d), 138.3 (s), 140.7 (s), 157.9 (s), 160.3 (CO), 170.3 (CO). Anal calcd for C₂₁H₂₀N₂O₅S: C, 61.15; H, 4.89; N, 6.79. Found: C, 61.52; H, 4.62; N, 6.85.

Ethyl 1-benzyl-7-(phenylsulfonyl)-1,4,5,9-tetrahydro-8H-pyrrolo[3,2-h]quinolin-8-one-2-carboxylate (9k).

This product was obtained from the reaction of 7j with phenylsulfonylacetonitrile after 24 h reflux. The crude precipitate was purified by column chromatography (Sepacore Büchi) using DCM/AcOEt 9:1 as eluent. Brown solid; yield 42%; mp: 167-169° C.; IR: ν 3309 (NH), 1701 (CO), 1637 (CO) cm⁻¹; ¹H NMR: δ 1.21 (3H, t, J=7.1 Hz, CH₃), 2.70 (2H, t, J=7.3 Hz, CH₂), 2.90 (2H, t, J=7.3 Hz, CH₂), 4.17 (2H, q, J=7.1 Hz, CH₂), 6.43 (2H, s, CH₂), 6.88 (1H, s, H-3), 6.91 (2H, d, J=7.2 Hz, H-2″ and H-6″), 7.09-7.23 (3H, m, Ar), 7.55-7.72 (3H, m, Ar), 7.95 (2H, d, J=7.8 Hz, H-2′ and H-6′), 8.14 (1H, s, H-6), 11.93 (1H, s, NH); ¹³C NMR: δ 14.1 (q), 20.8 (t), 27.2 (t), 48.4 (t), 59.9 (t), 115.9 (d), 117.3 (s), 123.9 (s), 125.6 (s), 126.0 (d), 126.6 (d), 127.5 (s), 127.8 (d), 128.2 (d), 129.0 (d), 130.7 (s), 133.4 (d), 138.1 (d), 139.4 (s), 140.7 (s), 150.4 (s), 157.9 (CO), 160.1 (CO). Anal calcd for C₂H₂₄N₂O₅S: C, 66.38; H, 4.95; N, 5.73. Found: C, 66.18; H, 5.12; N, 5.60.

Ethyl 1-(p-methyl-benzyl)-7-(phenylsulfonyl)-1,4,5,9-tetrahydro-8H-pyrrolo[3,2-h] quinolin-8-one-2-carboxylate (9l).

This product was obtained from the reaction of 7k with phenylsulfonylacetonitrile after 24 h reflux. The crude precipitate was purified by column chromatography (Sepacore Bilchi) using DCM/AcOEt 9:1 as eluent. Brown solid; yield 35%; mp: 98-100° C.; IR: ν 3299 (NH), 1701 (CO), 1664 (CO) cm⁻¹; ¹H NMR: δ 1.22 (3H, t, J=7.1 Hz, CH₃), 2.16 (3H, s, CH₃), 2.69 (2H, t, J=7.4 Hz, CH₂), 2.85 (2H, t, J=7.4.Hz, CH₂), 4.19 (2H, q, J=7.1 Hz, CH₂), 6.37 (2H, s, CH₂), 6.78 (2H, d, J=7.9 Hz, H-3″ and H-5″), 6.87 (1H, s, H-3), 6.98 (2H, d, J=7.9 Hz, H-2″ and H-6″), 7.59-7.69 (3H, m, Ar), 7.94 (2H, d, J=7.5 Hz, H-2′ and H-6′), 8.12 (1H, s, H-6), 11.92 (1H, s, NH); ¹³C NMR: δ 14.1 (q), 20.5 (q), 20.8 (t), 27.2 (t), 59.9 (t), 64.9 (t), 99.5 (d), 115.9 (d), 117.0 (s), 123.7 (s), 125.9 (d), 126.3 (s), 127.5 (s), 127.9 (d), 128.8 (d), 128.9 (s), 129.0 (d), 130.7 (s), 133.4 (d), 135.7 (s), 136.4 (s), 140.6 (s), 157.9 (CO), 160.1 (CO). Anal calcd for C₂₈H₂₆N₂O₅S:C, 66.91; H, 5.21; N, 5.57. Found: C, 66.85; H, 5.54; N, 5.42.

Ethyl 1-(p-methoxy-benzyl)-7-(phenylsulfonyl)-1,4,5,9-tetrahydro-8H-pyrrolo[3,24] quinolin-8-one-2-carboxylate (9m).

This product was obtained from the reaction of 71 with phenylsulfonylacetonitrile after 22 h reflux. The crude precipitate was purified by column chromatography (Sepacore Btichi) using DCM/AcOEt 9:1 as eluent. Brown solid; yield 37%; mp: 98-100° C.; IR: ν 3297 (NH), 1701 (CO), 1664 (CO) cm⁻¹; ¹H NMR: δ 1.23 (3H, t, J=7.1 Hz, CH₃), 2.69 (2H, t, J=7.2 Hz, CH₂), 2.85 (2H, t, J=7.2 Hz, CH₂), 3.40 (3H, s, CH₃), 4.21 (2H, q, J=7.1 Hz, CH₂), 6.34 (2H, s, CH₂), 6.74 (2H, d, J=7.2 Hz, H-3″ and H-5″), 6.86 (1H, s, H-3), 6.90 (2H, d, J=7.2 Hz, H-2″ and H-6″), 7.57-7.69 (3H, m, Ar), 7.95 (2H, d, J=6.8 Hz, H-2′ and H-6′), 8.14 (1H, s, H-6), 11.95 (1H, s, NH); ¹³C NMR: δ 14.1 (q), 20.8 (t), 27.2 (t), 47.7 (t), 54.9 (q), 64.9 (t), 99.5 (d), 113.6 (d), 115.9 (d), 117.2 (s), 124.1 (s), 127.5 (d), 127.6 (s), 127.9 (d), 129.0 (d), 129.7 (s), 130.6 (s), 131.3 (s), 131.6 (s), 133.4 (d), 138.1 (s), 140.6 (s), 159.1 (CO), 160.2 (CO). Anal calcd for C₂₈H₂₆N₂O₆S:C, 64.85; H, 5.05; N, 5.40. Found: C, 64.70; H, 5.42; N, 5.53.

7-(Phenylsulfonyl)-1,4,5,9-tetrahydro-8H-pyrrolo[3,2-h]quinolin-8-one (9b).

To a suspension of 9a (70 mg, 0.145 mmol) in ethanol (20 ml), KOH (0.06 g, 1 mmol) as added and the reaction mixture was refluxed for 1 h. After cooling, the reaction mixture was made acid with 6N HCl. The yellow solid formed was filtered, air dried and purified by column chromatography (Sepacore Bachi) using DCM/AcOEt 9:1 as eluent. Yield 90%; m.p.: 410° C.; IR: ν 3365 (NH), 3255 (NH), 1647 (CO) cm⁻¹; ¹H NMR: δ 2.64-2.83 (4H, m, 2×CH₂), 6.13 (1H, t, J=2.4 Hz, H-3), 7.15 (1H, d, J=2.4 Hz, H-2), 7.52-7.69 (3H, m, Ar), 7.96 (2H, d, J=8.0 Hz, H-2′ and H-6′), 8.08 (1H, s, H-6), 11.28 (1H, s, NH), 11.97 (1H, s, NH); ¹³C NMR: δ 21.2 (t), 25.9 (t), 99.5 (d), 108.8 (d), 118.8 (s), 119.9 (s), 125.6 (d), 127.6 (d), 128.6 (d), 129.6 (s), 132.8 (d), 141.2 (s), 142.4 (s), 142.9 (s), 156.8 (CO). Anal calcd for C₁₇H₁₄N₂O₃S:C, 62.56; H, 4.32; N, 8.58. Found: C, 62.74; H, 4.15; N, 8.40.

### EXAMPLE 2

The evaluation of phototoxic activity was carried out in some human tumour cell lines: T-cell leukaemia (Jurkat), chronic myeloid leukaemia (K-562), colon adenocarcinoma (LoVo) and breast adenocarcinoma (MCF-7). Cellular survival experiments were carried out without irradiation to verify an eventual toxicity of photosensitizer in the dark, and then after two times of irradiation (10 and 15 min of UV-A, which correspond to 2.5 and 3.75 J/cm², respectively), using lamps which emit mainly at 365 nm. Cell survival was checked after 72 h of compound incubation or after 72 h from UVA irradiation by Mosmann's MTT reduction test (3-[4,5-dimethyltiazol-2yl] 2,5-diphenyl tetrazolium bromide), (T. Mossman, Rapid colorimetric assay for cellular growth and survival: application to proliferation and cytotoxicity assays, J. Immunol. Meths. 1983, 65, 55-63). Cells incubated with compounds in the dark presented a cellular survival comparable to that of controls. In Table 2, there are GI₅₀ values, that is the compound concentration required for 50% cell survival, for each cell line and for every time of irradiation.

TABLE 2

| | ^{a}GI₅₀ (μM) | | | | | | | |
|---|---|---|---|---|---|---|---|---|
| | Jurkat | | K-562 | | LoVo | | MCF-7 | |
| | ^{b}2.5 | 3.75 | 2.5 | 3.75 | 2.5 | 3.75 | 2.5 | 3.75 |
| 9a | >20 | >20 | >20 | >20 | >20 | >20 | >20 | >20 |
| 9b | >20 | >20 | >20 | >20 | >20 | >20 | >20 | >20 |
| 9c | >20 | >20 | >20 | >20 | >20 | >20 | >20 | >20 |
| 9d | >20 | >20 | >20 | >20 | >20 | >20 | >20 | >20 |
| 9e | 1.3 ± 0.2 | 1.1 ± 0.1 | 4.8 ± 0.5 | 3.3 ± 0.5 | 2.6 ± 0.2 | 2.2 ± 0.2 | 4.0 ± 0.6 | 1.7 ± 0.7 |
| 9f | >20 | >20 | >20 | >20 | >20 | >20 | >20 | >20 |
| 9g | 1.6 ± 0.1 | 1.2 ± 0.2 | 2.7 ± 0.2 | 2.4 ± 0.1 | 2.7 ± 0.1 | 2.3 ± 0.2 | 3.8 ± 0.5 | 2.4 ± 0.2 |
| 9h | 1.6 ± 0.2 | 1.2 ± 0.2 | 3.1 ± 0.4 | 2.5 ± 0.2 | 2.8 ± 0.3 | 2.0 ± 0.2 | 4.2 ± 0.7 | 1.8 ± 0.1 |
| 9i | 3.5 ± 0.4 | 2.5 ± 0.2 | 6.1 ± 0.2 | 5.4 ± 0.6 | 5.5 ± 0.2 | 4.6 ± 0.4 | 6.1 ± 0.2 | 4.3 ± 0.7 |
| 9j | 0.7 ± 0.1 | 0.5 ± 0.1 | 1.0 ± 0.1 | 0.9 ± 0.1 | 1.1 ± 0.1 | 1.0 ± 0.1 | 1.2 ± 0.1 | 1.0 ± 0.1 |
| 9k | 0.8 ± 0.1 | 0.6 ± 0.1 | 0.9 ± 0.1 | 0.6 ± 0.1 | 0.9 ± 0.1 | 0.8 ± 0.1 | 0.9 ± 0.1 | 0.8 ± 0.1 |
| 9l | 1.5 ± 0.2 | 1.2 ± 0.1 | 1.2 ± 0.3 | 1.0 ± 0.1 | 1.6 ± 0.2 | 1.0 ± 0.2 | 1.7 ± 0.3 | 1.5 ± 0.2 |
| 9m | 0.8 ± 0.1 | 0.7 ± 0.1 | 1.0 ± 0.1 | 0.9 ± 0.1 | 1.1 ± 0.1 | 1.0 ± 0.2 | 1.6 ± 0.2 | 1.4 ± 0.2 |
| ^{c}Ang | 1.0 ± 0.2 | 0.9 ± 0.1 | 1.2 ± 0.1 | 1.0 ± 0.1 | 3.6 ± 0.4 | 1.5 ± 0.3 | 4.4 ± 0.5 | 1.5 ± 0.2 |

^{a}Values are espresse as mean ± SEM of at least 3 independent experiments
^{b}UV-A doeses esprexed as J/cm².
^{c}Ang = angelicin, reference drug.

8 of the 13 new synthesised compounds resulted phototoxic with GI₅₀ lower than 10 μM in all tested cell lines. In all cases, the phototoxic effect was dependent both on compound concentration and UVA dose. 9j, 9k e 9m resulted the most active compounds with submicromolar GI₅₀ comparable if not lower than the reference furocoumarin, angelicin, especially in adhesion cell lines. These three compounds were selected as archetypes to better analyse the photoinduced cellular death mode and their mechanism of action.

### EXAMPLE 3

The mode of cell death induction (necrosis or apoptosis) was evaluated. It is better that a chemotherapic agent induces cell death by apoptosis since it does not cause an intense inflammatory response as there is no release of cytoplasmatic components in the extracellular space.

Flow cytometer was used to evaluate the induction of cellular death. A first experiment was performed to assess an early apoptotic event, such as the exposure of macrophage recognition and phagocytosis antigens by cells which decide to die. One of these signals is phosphatidylserine (PS), a phospholipid which is normally found in the inner leaflet of plasmatic membrane, but which is translocated to the outer one during apoptosis. The Annexin V-FITC/PI cytofluorimetric test was used to verify phosphatidylserine exposure after 30 min and 2 h from irradiation with 9j, 9k and 9m (I. Vermes, C. Haanen, H. Steffens-Nakken, C. Reutelingsperger, A novel assay for apoptosis: Flow cytometric detection of phosphatidylserine expression on early apoptotic cells using fluorescein labelled Annexin V, J. Immunol. Meths. 1995, 184, 39-51).

A significant increase of cells in early apoptotic phase with respect to control can be observed just after 30 min from irradiation in the presence of compounds. After 2 h from irradiation, most cells are already in late apoptotic phase, losing their plasmatic membrane integrity. From this test, a cell death mainly by apoptosis can be hypothesised.

### EXAMPLE 4

Other two tests were performed to have more information about the mechanism of action and to evaluate which organelles were involved in this process. The complex role of mitochondria in apoptosis was identified by different biochemical studies which demonstrated various mitochondrial proteins are able to directly activate cellular apoptotic programs. (X. Wang, The expanding role of mitochondria in apoptosis, Gen. Develop. 2001, 15, 2922-2933). An eventual mitochondrial involvement in apoptosis induction was evaluated by such a parameter of mitochondrial dysfunction as the loss of mitochondrial membrane potential. Mitochondrial membrane potential of Jurkat cells was monitored by JC-1 probe after their irradiation in the presence of 9j, 9k and 9m (S. Salvioli, A. Ardizzoni, C. Franceschi, A. Cossarizza, JC-1, but not DiOC₆(3) or rhodamine 123, is a reliable fluorescent probe to assess AT changes in intact cells: implications for studies on mitochondrial functionality during apoptosis, FEBS Lett. 1997, 411, 77-82).

Mitochondria are surely implicated in cell death induction as an increase of cell percentage with collapsed mitochondrial potential was detected even after 2 h from irradiation (above all with 9k and 9m) with respect to control. This latter increased again after 4 h from irradiation.

Even other organelles such as lysosomes can be involved in the ordered propagation of apoptotic events. The method used to evaluate the lysosomial involvement of apoptosis induction was the acridine orange re-uptake by FACS (M. Zhao, J. W. Eaton, U. T. Brunk, Protection against oxidant-mediated lysosomial rupture: a new anti-apoptotic activity of Bcl-2?, FEBS Lett. 2000, 485, 104-108).

Even lysosomes are involved in apoptosis induction as an increase of cells with lysosomial dysfunction was observed after irradiation of Jurkat cells in the presence of all compounds with respect to control.

### EXAMPLE 5

Some DNA photodamage experiments were performed to better investigate their mechanism of action as such target is so important for furocoumarin derivatives.

After having assessed a weak affinity toward this macromolecule in the dark by such spectroscopic techniques as linear dichroism and fluorimetric titrations, some plasmid (pBR322) strand break experiments were carried out to check an eventual DNA photodamage by compounds. The formation of open circular or linear DNA from a supercoiled plasmid DNA was monitored by the separation of the three forms using agarose gel horizontal electrophoresis. Beside the formation of frank strand breaks, oxidative damages to purine and pyrimidine bases were also evaluated, incubating the irradiated mixture with the repair enzyme Fpg (Formamidopyrimidine glycosylase) and Endo III (Endonuclase III), respectively. (B. Epe, M. Pflaum, S. Boiteux, DNA damage induced by photosensitizers in cellular and cell-free systems, Mut. Res. 1993, 299, 135-145; T. A. Ciulla, J. R. Van Camp, E. Rosenfeld, I. Kochevar, Photosensitization of single-strand breaks in pBR322 DNA by Rose Bengal, Photochem. Photobiol. 1989, 49, 293-298).

Compounds 9j, 9k and 9m do not induce DNA photodamage: in fact, nor DNA photocleavage activity nor oxidative damages in DNA bases were checked.

New furocoumarin analogues with pyrrolo[3,2-h]quinolinone nucleus were synthesised with the aim of obtaining new potential photochemotherapic agents with minor adverse effects than psoralens. Many of the new synthesised molecules demonstrated in vitro phototoxicity in many human tumour cell lines after UVA irradiation. Their G1₅₀ values were in the range between 6.1 and 0.5 gM. The most active compounds were selected (9j, 9k and 9m) and they presented phototoxicity comparable if not higher to the reference compound, angelicin.

Pyrrolo[3,2-h]quinolinones induce cell death mainly by apoptosis as psoralens (M. Canton, S. Caffieri, F. Dall'Acqua, F. Di Lisa, PUVA-induced apoptosis involves mitochondrial dysfunction caused by the opening of the permeability transition pore, FEBS Lett. 2002, 522, 168-172; G. Viola, E. Fortunato, L. Cecconet, S. Disarò, G. Basso, Induction of apoptosis in Jurkat cells by photoexcited psoralen derivatives: Implication of mitochondrial dysfunctions and caspases activaction, Toxicol. Vitro 2007, 21, 211-216) and with the involvement of both mitochondria and lysosomes.

A potential interaction with DNA was also evaluated as this macromolecule represents such important target for the antiproliferative activity of PUVA therapy. Those compounds do not show DNA affinity. Moreover, no DNA photodamage was observed by a series of photocleavage experiments: nor the formation of frank strand breaks nor the presence of oxidative damages at base level.

As a consequence, from all exposed so far it is evident that pyrrolo[3,2-h]quinolinones show these advantages:
Elevated photoantiproliferative activity of new structures with a potential use in the treatment of neoplastic diseases.
Photoinduction of cellular death by apoptosis.
Absence of genotoxicity in vitro and so of the long term side effects of psoralens (mutagenesis and increased risk of cutaneous tumour) which limit the use of PUVA therapy.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

1. A drug with the following structure: 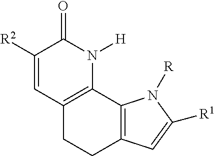
or its pharmaceutical acceptable derivative in which
R is independently a hydrogen, a small alkyl group, a phenyl group, a substituted or non-substituted benzyl group and a phenylsulfonic group, R¹ is independently a hydrogen or a carboxyl acid ester, R² is independently a cyano or a phenylsulfonic group.

2. The drug according to claim 1 or its pharmaceutical acceptable active derivative in which at least one between R or R¹ is a hydrogen.

3. The drug according to claim 1 or its pharmaceutical acceptable active derivative in which R¹ is an ethylic ester of carboxilic acid.

4. The drug according to claim 3 or its pharmaceutical acceptable active derivative in which R is a benzyl group substituted with a methyl or methoxy groups.

5. The drug according to claim 3 or its pharmaceutical acceptable active derivative in which R is a substituted or non-substituted benzyl group.

6. The drug according to claim 3 or its pharmaceutical acceptable active derivative in which R is a methyl group.

7. The drug according to claim 6 used in the treatment of tumors or every kind of hyperproliferative disease.

8. The drug according to claim 7 administered after UVA radiation activation for the cure of PUVA-treated diseases.

9. The drug according to claim 8 useful for the treatment of neoplasia with the photochemotherapy strategy (drug+UVA light); the innovation consists of the fact that in the classical PUVA there are important side effects, such as the risk of skin cancer, whereas for the drugs of this patent the pharmacologic strategy is analogue (use of photochemotherapy) but their activity does not cause severe adverse effects (genotoxicity and risk of skin cancer) as they do not induce DNA photodamage.
